(19) United States
(12) Patent Application Publication
(10) Pub. No.: US 2019/0247489 A1
(43) Pub. Date: Aug. 15, 2019

(54) ADJUVANTED INFLUENZA B VIRUS VACCINES FOR PEDIATRIC PRIMING

(71) Applicant: Seqirus UK Limited, Berkshire (GB)

(72) Inventor: Theodore TSAI, Cambridge, MA (US)

(21) Appl. No.: 16/193,024

(22) Filed: Nov. 16, 2018

Related U.S. Application Data
(63) Continuation of application No. 14/352,955, filed on Apr. 18, 2014, now abandoned.
(60) Provisional application No. 61/627,995, filed on Oct. 20, 2011.

(51) Int. Cl. A61K 39/145; A61K 39/12; A61K 9/107

(52) U.S. Cl. CPC A61K 39/145; A61K 39/12; A61K 2039/55566; C12N 2760/16134; A61K 9/107

(57) ABSTRACT

The influenza B strain is epidemiologically relevant in the pediatric population. Immunogenic priming of children with influenza B vaccine adjuvanted with an oil-in-water emulsion primes an immune response to a booster vaccine comprising influenza B virus antigen from a differing strain or lineage, irrespective of whether the booster comprises an adjuvant.

### TECHNICAL FIELD

This invention is in the field of adjuvanted vaccines for protecting against influenza virus infection in children.

### BACKGROUND ART

Influenza vaccines currently in general use are described in chapters 17 & 18 of reference 1. They are based on live virus or inactivated virus, and inactivated vaccines can be based on whole virus, ‘split’ virus or on purified surface antigens (including haemagglutinin and neuraminidase).

The burden of influenza in healthy young children has been increasingly recognized along with new studies on the medical [2-7] and the socioeconomic [8] impact of influenza. Moreover, children have the highest attack rates of influenza during epidemic periods, and transmit influenza viruses in the community to the high risk groups [8,9].

The American Advisory Committee on Immunization Practices (ACIP) in 2006 recommended annual influenza vaccination for all children aged 6-59 months, because children aged 6-23 months are at substantially increased risk for influenza-related hospitalizations [2-7] and children aged 24-59 months are at increased risk for influenza-related clinic and emergency department visits [6]. In July 2008 the ACIP further extended the recommendation for seasonal influenza vaccination in adolescents aged 5 to 18 years [10]. In Europe, some countries have issued similar recommendations, although the European CDC has taken a more restricted position with regard to universal immunization of young children, noting that efficacy in children under 24 months of age has been insufficiently documented and might be as low as 37% [11]. A Cochrane analysis stated that “the field efficacy of influenza vaccine in young children is not different from placebo” [12].

In addition to modest efficacy, conventional vaccines do not appear to induce satisfactory protective antibodies in unprimed children, especially the very young ones. More specifically, conventional vaccines generally show lower immunogenicity against the influenza B strain than against influenza A strains [13,14]. ACIP has since 2004 recommended a two-dose vaccination regimen in immunologically naive very young children, but more recently such recommendation has been extended to children aged up to 8 years of age, because of the accumulating evidence indicating that 2 doses are required for protection in this population [15].

An additional problem in immunizing children against influenza comes from ‘antigenic drift’. Influenza viruses routinely undergo intense selection to evade the host immune system, resulting in genetic variation and the generation of novel strains (‘antigenic drift’). It has been suggested that antigenic drift is associated with a more severe and early onset of influenza epidemic, since the level of pre-existing immunity to the drifted strain is reduced to the drifted strain [16]. While all three virus strains currently included in seasonal influenza vaccines are subject to antigenic drift, the A/H3N2 strain is known to drift more frequently and new variants tend to replace old ones [17,18].

The pace of antigenic drift can exceed the pace of vaccine manufacture. When a vaccine is released, therefore, the vaccine strains may no longer be a good match for the circulating strains. A vaccine mismatch can result in a significant excess of influenza-related mortality, since vaccine effectiveness is reduced [19]. Vaccine mismatch is a potentially larger problem in the most influenza susceptible populations, particularly in young children who do not have pre-existing immunity against any influenza viruses. This was shown more recently in the 2003/2004 season by the emergence of a drifted mismatch strain (A/Fujian, H3N2), which was not included in the vaccine, and resulted in 3 times as many children being hospitalized in intensive care in California, compared with the previous season [20]. In contrast to young children, the elderly at least have a significant history of prior exposure to circulating influenza strains, which offers them some degree of cross protection. Drifted influenza strains which emerge after vaccine recommendations are finalized, as occurred in 1997 and 2003, are a significant threat to vaccine-naive young children.

It is an object of the invention to provide influenza vaccines that are effective in children, that give adequate influenza B virus immunogenicity (to induce an adequate immune response), that give useful protection against common circulating influenza viruses, and/or that are effective in children against influenza B virus strains.

### SUMMARY OF THE INVENTION

It has now been found that an influenza vaccine comprising an influenza B virus antigen and adjuvanted with a sub-micron oil-in-water emulsion primes the immune system so that, compared to an equivalent unadjuvanted vaccine, it is better able to respond to subsequent exposure to influenza B antigens from heterologous strains and in particular from strains in different lineages.

Thus the invention provides a method for immunizing a child, comprising (i) administering to the child an immunogenic composition comprising an antigen from a first influenza B virus and an adjuvant comprising an oil-in-water emulsion, then (ii) administering to the child an immunogenic composition comprising an antigen from a second influenza B virus and, optionally, an adjuvant comprising an oil-in-water emulsion; wherein the first influenza B virus and the second influenza B virus are different strains (and, preferably, are in different lineages).

The invention also provides a method for re-immunizing a child, comprising administering to the child a second immunogenic composition comprising an antigen from a second influenza B virus; wherein the child has been pre-immunized with a first immunogenic composition comprising an antigen from a first influenza B virus and an adjuvant comprising an oil-in-water emulsion, wherein the first influenza B virus and the second influenza B virus are in different lineages.

The invention also provides first and second immunogenic compositions, individually comprising antigen from first and second influenza B virus strains in different lineages, for use in a method for immunizing a child, comprising (i) administering to the child the first immunogenic composition, comprising an antigen from the first influenza B virus and an adjuvant comprising an oil-in-water emulsion, then (ii) administering to the child the second immunogenic composition, comprising an antigen from the second influenza B virus.

The invention also provides a second immunogenic composition comprising an antigen from a second influenza B virus strain, for use in a method for re-immunizing a child, comprising administering to the child the second immunogenic composition; wherein the child has been pre immunized with a first immunogenic composition comprising an antigen from a first influenza B virus and an adjuvant comprising an oil-in-water emulsion, wherein the first influenza B virus and the second influenza B virus are in different lineages.

The invention also provides the use of antigen from a second influenza B virus strain in the manufacture of an influenza vaccine for re-immunizing a child, wherein (i) the child has been pre-immunized with antigen from a first influenza B virus and an adjuvant comprising an oil-in-water emulsion, and (ii) the first influenza B virus and the second influenza B virus are in different lineages.

The child being immunized or re-immunized may be aged between 0 months and 36 months e.g. between 6 months and 35 months, between 6 months and 30 months, between 6 months and 24 months, between 6 months and 23 months (all inclusive) Immunization is ideal after a child is 6 months old but before their third birthday, as described in more detail below. The invention can also be used with older children e.g. up to 72 months of age. Thus the child may be between 6 and 72 months old, between 36 and 72 months old, etc. and so a vaccine may be administered before a child's sixth birthday.

An adjuvanted vaccine that can be used according to the invention is the FLUAD™ product, which is already available but is approved for use only in elderly subjects i.e. subjects at least 65 years of age (or, in some regions, at least 60 years of age). The adjuvant in this vaccine is a sub-micron oil-in-water emulsion known as MF59. The adjuvant in FLUAD™ helps to overcome the age-related immuno-senescence seen in the elderly.

### DETAILED DESCRIPTION

### The Influenza Virus Antigen

The invention uses an influenza virus antigen, typically comprising hemagglutinin, to immunize a child. The antigen will typically be prepared from influenza virions but, as an alternative, antigens such as haemagglutinin can be expressed in a recombinant host (e.g. in an insect cell line using a baculovirus vector) and used in purified form [21,22]. In general, however, antigens will be from virions.

The antigen may take the form of a live virus or, more preferably, an inactivated virus. Chemical means for inactivating a virus include treatment with an effective amount of one or more of the following agents: detergents, formaldehyde, formalin, β-propiolactone, or UV light. Additional chemical means for inactivation include treatment with methylene blue, psoralen, carboxyfullerene (C60) or a combination of any thereof. Other methods of viral inactivation are known in the art, such as for example binary ethylamine, acetyl ethyleneimine, or gamma irradiation. The INFLEXAL™ product is a whole virion inactivated vaccine.

Where an inactivated virus is used, the vaccine may comprise whole virion, split virion, or purified surface antigens (including hemagglutinin and, usually, also including neuraminidase).

An inactivated but non-whole cell vaccine (e.g. a split virus vaccine or a purified surface antigen vaccine) may include matrix protein, in order to benefit from the additional T cell epitopes that are located within this antigen. Thus a non-whole cell vaccine (particularly a split vaccine) that includes haemagglutinin and neuraminidase may additionally include M1 and/or M2 matrix protein. Useful matrix fragments are disclosed in reference 23. Nucleoprotein may also be present.

Virions can be harvested from virus-containing fluids by various methods. For example, a purification process may involve zonal centrifugation using a linear sucrose gradient solution that includes detergent to disrupt the virions. Antigens may then be purified, after optional dilution, by diafiltration.

Split virions are obtained by treating purified virions with detergents and/or solvents to produce subvirion preparations, including the ‘Tween-ether’ splitting process. Methods of splitting influenza viruses are well known in the art e.g. see refs. 24-29, etc. Splitting of the virus is typically carried out by disrupting or fragmenting whole virus, whether infectious or non-infectious with a disrupting concentration of a splitting agent. The disruption results in a full or partial solubilisation of the virus proteins, altering the integrity of the virus. Preferred splitting agents are non-ionic and ionic (e.g. cationic) surfactants. Suitable splitting agents include, but are not limited to: ethyl ether, polysorbate 80, deoxycholate, tri-N-butyl phosphate, alkylglycosides, alkylthioglycosides, acyl sugars, sulphobetaines, betaines, polyoxyethylenealkylethers, N,N-dialkyl-Glucamides, Hecameg, alkylphenoxy-polyethoxyethanols, quaternary ammonium compounds, sarcosyl, CTABs (cetyl trimethyl ammonium bromides), tri-N-butyl phosphate, Cetavlon, myristyltrimethylammonium salts, lipofectin, lipofectamine, and DOT-MA, the octyl- or nonylphenoxy polyoxyethanols (e.g. the Triton surfactants, such as Triton X-100 or Triton N101), nonoxynol 9 (NP9) Sympatens-NP/090,) polyoxyethylene sorbitan esters (the Tween surfactants), polyoxyethylene ethers, polyoxyethlene esters, etc. One useful splitting procedure uses the consecutive effects of sodium deoxycholate and formaldehyde, and splitting can take place during initial virion purification (e.g. in a sucrose density gradient solution). Thus a splitting process can involve clarification of the virion-containing material (to remove non-virion material), concentration of the harvested virions (e.g. using an adsorption method, such as CaHPO₄ adsorption), separation of whole virions from non-virion material, splitting of virions using a splitting agent in a density gradient centrifugation step (e.g. using a sucrose gradient that contains a splitting agent such as sodium deoxycholate), and then filtration (e.g.

ultrafiltration) to remove undesired materials. Split virions can usefully be resuspended in sodium phosphate-buffered isotonic sodium chloride solution. The BEGRIVAC™, FLUARIX™, FLUZONE™ and FLUSHIELD™ products are split vaccines.

Purified surface antigen vaccines comprise the influenza surface antigens haemagglutinin and, typically, also neuraminidase. Processes for preparing these proteins in purified form are well known in the art. The FLUVIRIN™, AGRIPPAL™ and INFLUVAC™ products are subunit vaccines.

Another form of inactivated influenza antigen is the virosome [30] (nucleic acid free viral-like liposomal particles). Virosomes can be prepared by solubilization of influenza virus with a detergent followed by removal of the nucleocapsid and reconstitution of the membrane containing the viral glycoproteins. An alternative method for preparing virosomes involves adding viral membrane glycoproteins to excess amounts of phospholipids, to give liposomes with viral proteins in their membrane. The invention can be used to store bulk virosomes. as in the INFLEXAL V™ and INVAVAC™ products. In some embodiments, the influenza antigen is not in the form of a virosome.

The influenza virus may be attenuated. The influenza virus may be temperature-sensitive. The influenza virus may be cold-adapted. These three features are particularly useful when using live virus as a vaccine antigen.

HA is the main immunogen in current inactivated influenza vaccines, and vaccine doses are standardised by reference to HA levels, typically measured by SRID. Existing vaccines typically contain about 15 μg of HA per strain, although lower doses can be used e.g. for children, or in pandemic situations, or when using an adjuvant. Fractional doses such as ½ (i.e. 7.5 μg HA per strain), ¼ and ⅛ have been used, as have higher doses (e.g. 3× or 9× doses [31,32]). Thus vaccines may include between 0.1 and 150 μg of HA per influenza strain, preferably between 0.1 and 50 μg e.g. 0.1-20 μg, 0.1-15 μg, 0.1-10 μg, 0.1-7.5 μg, 0.5-5μg, etc. Particular doses include e.g. about 45, about 30, about 15, about 10, about 7.5, about 5, about 3.8, about 1.9, about 1.5, etc. per strain. A dose of 7.5 μg per strain is ideal for use in children.

For live vaccines, dosing is measured by median tissue culture infectious dose (TCID₅₀) rather than HA content, and a TCID50 of between 10⁶ and 10⁸ (preferably between 10⁶⁵-10⁷⁵) per strain is typical.

Influenza virus strains for use in vaccines change from season to season. In the current inter-pandemic period, vaccines typically include two influenza A strains (H1N1 and H3N2) and one influenza B strain, and trivalent vaccines are typical for use with the invention. Compositions of the invention comprise antigen from influenza B virus and optionally comprise antigen from at least one influenza A virus. Where the composition of the invention comprises antigen from influenza A virus(es), the invention may use seasonal and/or pandemic strains. Depending on the season and on the nature of the antigen included in the vaccine, the invention may include (and protect against) one or more of influenza A virus hemagglutinin subtypes H1, H2, H3, H4, H5, H6, H7, H8, H9, H10, H11, H12, H13, H14, H15 or H16. The vaccine may additionally include neuraminidase from any of NA subtypes N1, N2, N3, N4, N5, N6, N7, N8 or N9.

The invention can thus be used with pandemic influenza A virus strains. Characteristics of a pandemic strain are: (a) it contains a new hemagglutinin compared to the hemagglutinins in currently-circulating human strains, i.e. one that has not been evident in the human population for over a decade (e.g. H2), or has not previously been seen at all in the human population (e.g. H5, H6 or H9, that have generally been found only in bird populations), such that the vaccine recipient and the general human population are immunologically naive to the strain's hemagglutinin; (b) it is capable of being transmitted horizontally in the human population; and (c) it is pathogenic to humans. Pandemic strains include, but are not limited to, H2, H5, H7 or H9 subtype strains e.g. H5N1, H5N3, H9N2, H2N2, H7N1 and H7N7 strains. Within the H5 subtype, a virus may fall into a number of clades e.g. clade 1 or clade 2. Six sub-clades of clade 2 have been identified with sub-clades 1, 2 and 3 having a distinct geographic distribution and are particularly relevant due to their implication in human infections.

Influenza B virus currently does not display different HA subtypes, but influenza B virus strains do fall into two distinct lineages. These lineages emerged in the late 1980s and have HAs which can be antigenically and/or genetically distinguished from each other [33]. Current influenza B virus strains are either B/Victoria/2/87-like or B/Yamagata/16/88-like. These strains are usually distinguished antigenically, but differences in amino acid sequences have also been described for distinguishing the two lineages e.g. B/Yamagata/16/88-like strains often (but not always) have HA proteins with deletions at amino acid residue 164, numbered relative to the ‘Lee40’ HA sequence [34]. The invention can be used with antigens from a B virus of either lineage.

Where a vaccine includes more than one strain of influenza, the different strains are typically grown separately and are mixed after the viruses have been harvested and antigens have been prepared. Thus a manufacturing process of the invention may include the step of mixing antigens from more than one influenza strain.

An influenza virus used with the invention may be a reassortant strain, and may have been obtained by reverse genetics techniques. Reverse genetics techniques [e.g. 35-39] allow influenza viruses with desired genome segments to be prepared in vitro using plasmids. Typically, it involves expressing (a) DNA molecules that encode desired viral RNA molecules e.g. from poll promoters or bacteriophage RNA polymerase promoters, and (b) DNA molecules that encode viral proteins e.g. from polll promoters, such that expression of both types of DNA in a cell leads to assembly of a complete intact infectious virion. The DNA preferably provides all of the viral RNA and proteins, but it is also possible to use a helper virus to provide some of the RNA and proteins. Plasmid-based methods using separate plasmids for producing each viral RNA can be used [40-42], and these methods will also involve the use of plasmids to express all or some (e.g. just the PB1, PB2, PA and NP proteins) of the viral proteins, with up to 12 plasmids being used in some methods. To reduce the number of plasmids needed, a recent approach [43] combines a plurality of RNA polymerase I transcription cassettes (for viral RNA synthesis) on the same plasmid (e.g. sequences encoding 1, 2, 3, 4, 5, 6, 7 or all 8 influenza A vRNA segments), and a plurality of protein-coding regions with RNA polymerase II promoters on another plasmid (e.g. sequences encoding 1, 2, 3, 4, 5, 6, 7 or all 8 influenza A mRNA transcripts). Preferred aspects of the reference 43 method involve: (a) PB1, PB2 and PA mRNA-encoding regions on a single plasmid; and (b) all 8 vRNA-encoding segments on a single plasmid. Including the NA and HA segments on one plasmid and the six other segments on another plasmid can also facilitate matters.

As an alternative to using poll promoters to encode the viral RNA segments, it is possible to use bacteriophage polymerase promoters [44]. For instance, promoters for the SP6, T3 or T7 polymerases can conveniently be used. Because of the species-specificity of poll promoters, bacteriophage polymerase promoters can be more convenient for many cell types (e.g. MDCK), although a cell must also be transfected with a plasmid encoding the exogenous polymerase enzyme.

In other techniques it is possible to use dual poll and polll promoters to simultaneously code for the viral RNAs and for expressible mRNAs from a single template [45,46].

Thus an influenza A virus may include one or more RNA segments from a A/PR/8/34 virus (typically 6 segments from A/PR/8/34, with the HA and N segments being from a vaccine strain, i.e.

a 6:2 reassortant). It may also include one or more RNA segments from a A/WSN/33 virus, or from any other virus strain useful for generating reassortant viruses for vaccine preparation. An influenza A virus may include fewer than 6 (i.e. 0, 1, 2, 3, 4 or 5) viral segments from an AA/6/60 influenza virus (A/Ann Arbor/6/60). An influenza B virus may include fewer than 6 (i.e. 0, 1, 2, 3, 4 or 5) viral segments from an AA/1/66 influenza virus (B/Ann Arbor/1/66). Typically, the invention protects against a strain that is capable of human-to-human transmission, and so the strain's genome will usually include at least one RNA segment that originated in a mammalian (e g in a human) influenza virus. It may include NS segment that originated in an avian influenza virus.

Strains whose antigens can be included in the compositions may be resistant to antiviral therapy (e.g. resistant to oseltamivir [47] and/or zanamivir), including resistant pandemic strains [48].

HA used with the invention may be a natural HA as found in a virus, or may have been modified. For instance, it is known to modify HA to remove determinants (e.g. hyper-basic regions around the cleavage site between HA1 and HA2) that cause a virus to be highly pathogenic in avian species, as these determinants can otherwise prevent a virus from being grown in eggs.

The viruses used as the source of the antigens can be grown either on eggs (e.g. specific pathogen free eggs) or on cell culture. The current standard method for influenza virus growth uses embryonated hen eggs, with virus being purified from the egg contents (allantoic fluid). More recently, however, viruses have been grown in animal cell culture and, for reasons of speed and patient allergies, this growth method is preferred.

The cell line will typically be of mammalian origin. Suitable mammalian cells of origin include, but are not limited to, hamster, cattle, primate (including humans and monkeys) and dog cells, although the use of primate cells is not preferred. Various cell types may be used, such as kidney cells, fibroblasts, retinal cells, lung cells, etc. Examples of suitable hamster cells are the cell lines having the names BHK21 or HKCC. Suitable monkey cells are e.g. African green monkey cells, such as kidney cells as in the Vero cell line [49-51]. Suitable dog cells are e.g. kidney cells, as in the CLDK and MDCK cell lines.

Thus suitable cell lines include, but are not limited to: MDCK; CHO; CLDK; HKCC; 293T; BHK; Vero; MRC-5; PER.C6 [52]; FRhL2; WI-38; etc. Suitable cell lines are widely available e.g. from the American Type Cell Culture (ATCC) collection [53], from the Coriell Cell Repositories [54], or from the European Collection of Cell Cultures (ECACC). For example, the ATCC supplies various different Vero cells under catalog numbers CCL-81, CCL-81.2, CRL-1586 and CRL-1587, and it supplies MDCK cells under catalog number CCL-34. PER.C6 is available from the ECACC under deposit number 96022940.

The most preferred cell lines are those with mammalian-type glycosylation. As a less-preferred alternative to mammalian cell lines, virus can be grown on avian cell lines [e.g. refs. 55-57], including cell lines derived from ducks (e.g. duck retina) or hens. Examples of avian cell lines include avian embryonic stem cells [55,58] and duck retina cells [56]. Suitable avian embryonic stem cells, include the EBx cell line derived from chicken embryonic stem cells, EB45, EB14, and EB14-074 [59]. Chicken embryo fibroblasts (CEF) may also be used. Rather than using avian cells, however, the use of mammalian cells means that vaccines can be free from avian DNA and egg proteins (such as ovalbumin and ovomucoid), thereby reducing allergenicity.

The most preferred cell lines for growing influenza viruses are MDCK cell lines [60-63], derived from Madin Darby canine kidney. The original MDCK cell line is available from the ATCC as CCL-34, but derivatives of this cell line may also be used. For instance, reference 60 discloses a MDCK cell line that was adapted for growth in suspension culture (‘MDCK 33016’, deposited as DSM ACC 2219). Similarly, reference 64 discloses a MDCK-derived cell line that grows in suspension in serum-free culture (‘B-702’, deposited as FERM BP-7449). Reference 65 discloses non-tumorigenic MDCK cells, including ‘MDCK-S’ (ATCC PTA-6500), ‘MDCK-SF101’ (ATCC PTA-6501), ‘MDCK-SF102’ (ATCC PTA-6502) and ‘MDCK-SF103’ (PTA-6503). Reference 66 discloses MDCK cell lines with high susceptibility to infection, including ‘MDCK.5F1’ cells (ATCC CRL-12042). Any of these MDCK cell lines can be used.

Virus may be grown on cells in adherent culture or in suspension. Microcarrier cultures can also be used. In some embodiments, the cells may thus be adapted for growth in suspension.

Cell lines are preferably grown in serum-free culture media and/or protein free media. A medium is referred to as a serum-free medium in the context of the present invention in which there are no additives from serum of human or animal origin. The cells growing in such cultures naturally contain proteins themselves, but a protein-free medium is understood to mean one in which multiplication of the cells occurs with exclusion of proteins, growth factors, other protein additives and non-serum proteins, but can optionally include proteins such as trypsin or other proteases that may be necessary for viral growth.

Cell lines supporting influenza virus replication are preferably grown below 37° C. [67] (e.g. 30-36° C., or at about 30° C., 31° C., 32° C., 33° C., 34° C., 35° C., 36° C.) during viral replication.

Methods for propagating influenza virus in cultured cells generally includes the steps of inoculating a culture of cells with an inoculum of the strain to be grown, cultivating the infected cells for a desired time period for virus propagation, such as for example as determined by virus titer or antigen expression (e.g. between 24 and 168 hours after inoculation) and collecting the propagated virus. The cultured cells are inoculated with a virus (measured by PFU or TCID₅₀) to cell ratio of 1:500 to 1:1, preferably 1:100 to 1:5, more preferably 1:50 to 1:10. The virus is added to a suspension of the cells or is applied to a monolayer of the cells, and the virus is absorbed on the cells for at least 60 minutes but usually less than 300 minutes, preferably between 90 and 240 minutes at 25° C. to 40° C., preferably 28° C. to 37° C. The infected cell culture (e.g. monolayers) may be removed either by freeze-thawing or by enzymatic action to increase the viral content of the harvested culture supernatants. The harvested fluids are then either inactivated or stored frozen. Cultured cells may be infected at a multiplicity of infection (“m.o.i.”) of about 0.0001 to 10, preferably 0.002 to 5, more preferably to 0.001 to 2. Still more preferably, the cells are infected at a m.o.i of about 0.01. Infected cells may be harvested 30 to 60 hours post infection. Preferably, the cells are harvested 34 to 48 hours post infection. Still more preferably, the cells are harvested 38 to 40 hours post infection. Proteases (typically trypsin) are generally added during cell culture to allow viral release, and the proteases can be added at any suitable stage during the culture e.g. before inoculation, at the same time as inoculation, or after inoculation [67].

In preferred embodiments, particularly with MDCK cells, a cell line is not passaged from the master working cell bank beyond 40 population-doubling levels.

The viral inoculum and the viral culture are preferably free from (i.e. will have been tested for and given a negative result for contamination by) herpes simplex virus, respiratory syncytial virus, parainfluenza virus 3, SARS coronavirus, adenovirus, rhinovirus, reoviruses, polyomaviruses, birnaviruses, circoviruses, and/or parvoviruses [68]. Absence of herpes simplex viruses is particularly preferred.

Where virus has been grown on a cell line then it is standard practice to minimize the amount of residual cell line DNA in the final vaccine, in order to minimize any oncogenic activity of the DNA.

Thus a vaccine composition prepared according to the invention preferably contains less than 10 ng (preferably less than 1 ng, and more preferably less than 100 pg) of residual host cell DNA per dose, although trace amounts of host cell DNA may be present.

Vaccines containing <10 ng (e.g. <1 ng, <100 μg) host cell DNA per 15 μg of haemagglutinin are preferred, as are vaccines containing <10 ng (e.g. <1 ng, <100 μg) host cell DNA per 0.25 ml volume.

Vaccines containing <10 ng (e.g. <1 ng, <100 μg) host cell DNA per 50 μg of haemagglutinin are more preferred, as are vaccines containing <10 ng (e.g. <1 ng, <100 pg) host cell DNA per 0.5 ml volume.

It is preferred that the average length of any residual host cell DNA is less than 500 bp e.g. less than 400 bp, less than 300 bp, less than 200 bp, less than 100 bp, etc.

Contaminating DNA can be removed during vaccine preparation using standard purification procedures e.g. chromatography, etc. Removal of residual host cell DNA can be enhanced by nuclease treatment e.g. by using a DNase. A convenient method for reducing host cell DNA contamination is disclosed in references 69 & 70, involving a two-step treatment, first using a DNase (e.g. Benzonase), which may be used during viral growth, and then a cationic detergent (e.g. CTAB), which may be used during virion disruption. Removal by β-propiolactone treatment can also be used.

Measurement of residual host cell DNA is now a routine regulatory requirement for biologicals and is within the normal capabilities of the skilled person. The assay used to measure DNA will typically be a validated assay [71,72]. The performance characteristics of a validated assay can be described in mathematical and quantifiable terms, and its possible sources of error will have been identified. The assay will generally have been tested for characteristics such as accuracy, precision, specificity. Once an assay has been calibrated (e.g. against known standard quantities of host cell DNA) and tested then quantitative DNA measurements can be routinely performed. Three main techniques for DNA quantification can be used: hybridization methods, such as Southern blots or slot blots [73]; immunoassay methods, such as the Threshold™ System [74]; and quantitative PCR [75]. These methods are all familiar to the skilled person, although the precise characteristics of each method may depend on the host cell in question e.g. the choice of probes for hybridization, the choice of primers and/or probes for amplification, etc. The Threshold™ system from Molecular Devices is a quantitative assay for picogram levels of total DNA, and has been used for monitoring levels of contaminating DNA in biopharmaceuticals [74]. A typical assay involves non-sequence-specific formation of a reaction complex between a biotinylated ssDNA binding protein, a urease-conjugated anti-ssDNA antibody, and DNA. All assay components are included in the complete Total DNA Assay Kit available from the manufacturer. Various commercial manufacturers offer quantitative PCR assays for detecting residual host cell DNA e.g. AppTec™ Laboratory Services, BioReliance™, Althea Technologies, etc. A comparison of a chemiluminescent hybridisation assay and the total DNA Threshold™ system for measuring host cell DNA contamination of a human viral vaccine can be found in reference 76.

### The Adjuvant

Compositions of the invention comprise an adjuvant, which can function to enhance the immune responses (humoral and/or cellular) elicited in a patient who receives the composition. Vaccine adjuvants for use with the invention comprise an oil-in-water emulsion.

Oil-in-water emulsions have been found to be particularly suitable for use in adjuvanting influenza virus vaccines. Various such emulsions are known, and they typically include at least one oil and at least one surfactant, with the oil(s) and surfactant(s) being biodegradable (metabolisable) and biocompatible. The oil droplets in the emulsion are generally less than 5 μm in diameter, and ideally the majority of oil droplets in the emulsion have a sub-micron diameter (e.g. at least 90% by number of the oil droplets have a sub-micron diameter), with these small sizes being achieved with a microfluidiser to provide stable emulsions. Droplets with a size less than 220 nm are preferred as they can be subjected to filter sterilization.

The emulsion can comprise oils such as those from an animal (such as fish) or vegetable source. Sources for vegetable oils include nuts, seeds and grains. Peanut oil, soybean oil, coconut oil, and olive oil, the most commonly available, exemplify the nut oils. Jojoba oil can be used e.g. obtained from the jojoba bean. Seed oils include safflower oil, cottonseed oil, sunflower seed oil, sesame seed oil and the like. In the grain group, corn oil is the most readily available, but the oil of other cereal grains such as wheat, oats, rye, rice, teff, triticale and the like may also be used. 6-10 carbon fatty acid esters of glycerol and 1,2-propanediol, while not occurring naturally in seed oils, may be prepared by hydrolysis, separation and esterification of the appropriate materials starting from the nut and seed oils. Fats and oils from mammalian milk are metabolizable and may therefore be used in the practice of this invention. The procedures for separation, purification, saponification and other means necessary for obtaining pure oils from animal sources are well known in the art. Most fish contain metabolizable oils which may be readily recovered. For example, cod liver oil, shark liver oils, and whale oil such as spermaceti exemplify several of the fish oils which may be used herein. A number of branched chain oils are synthesized biochemically in 5-carbon isoprene units and are generally referred to as terpenoids. Shark liver oil contains a branched, unsaturated terpenoids known as squalene, 2,6,10,15,19,23-hexamethyl-2,6,10,14,18,22-tetracosahexaene, which is particularly preferred herein (e.g. used at ≤11 mg per dose). Squalane, the saturated analog to squalene, is also a preferred oil. Fish oils, including squalene and squalane, are readily available from commercial sources or may be obtained by methods known in the art. Other preferred oils are the tocopherols (see below). Mixtures of oils can be used.

Surfactants can be classified by their ‘HLB’ (hydrophile/lipophile balance). Preferred surfactants of the invention have a HLB of at least 10, preferably at least 15, and more preferably at least 16. The invention can be used with surfactants including, but not limited to: the polyoxyethylene sorbitan esters surfactants (commonly referred to as the Tweens), especially polysorbate 20 and polysorbate 80; copolymers of ethylene oxide (EO), propylene oxide (PO), and/or butylene oxide (BO), sold under the DOWFAX™ tradename, such as linear EO/PO block copolymers; octoxynols, which can vary in the number of repeating ethoxy (oxy-1,2-ethanediyl) groups, with octoxynol-9 (Triton X-100, or t-octylphenoxypolyethoxyethanol) being of particular interest; (octylphenoxy)polyethoxyethanol (IGEPAL CA-630/NP-40); phospholipids such as phosphatidylcholine (lecithin); nonylphenol ethoxylates, such as the Tergitol™ NP series; polyoxyethylene fatty ethers derived from lauryl, cetyl, stearyl and oleyl alcohols (known as Brij surfactants), such as triethyleneglycol monolauryl ether (Brij 30); and sorbitan esters (commonly known as the SPANs), such as sorbitan trioleate (Span 85) and sorbitan monolaurate. Non-ionic surfactants are preferred. Preferred surfactants for including in the emulsion are Tween 80 (polyoxyethylene sorbitan monooleate or polysorbate 80), Span 85 (sorbitan trioleate), lecithin and Triton X-100.

Mixtures of surfactants can be used e.g. Tween 80/Span 85 mixtures. A combination of a polyoxyethylene sorbitan ester such as polyoxyethylene sorbitan monooleate (polysorbate 80) and an octoxynol such as t-octylphenoxypolyethoxyethanol (Triton X-100) is also suitable. Another useful combination comprises laureth 9 plus a polyoxyethylene sorbitan ester and/or an octoxynol.

Preferred amounts of surfactants (% by weight) are: polyoxyethylene sorbitan esters (such as polysorbate 80) 0.01 to 1%, in particular about 0.1%; octyl- or nonylphenoxy polyoxyethanols (such as Triton X-100, or other detergents in the Triton series) 0.001 to 0.1%, in particular 0.005 to 0.02%; polyoxyethylene ethers (such as laureth 9) 0.1 to 20%, preferably 0.1 to 10% and in particular 0.1 to 1% or about 0.5%.

Preferred emulsion adjuvants have an average droplets size of ≤1 μm e.g. ≤750 nm, ≤500 nm, ≤400 nm, ≤300 nm, ≤250 nm, ≤220 nm, ≤200 nm, or smaller These droplet sizes can conveniently be achieved by techniques such as microfluidisation.

Specific oil-in-water emulsion adjuvants useful with the invention include, but are not limited to:
A submicron emulsion of squalene, polysorbate 80, and sorbitan trioleate. These three components can be present at a volume ratio of 10:1:1 or a weight ratio of 39:47:47. The composition of the emulsion by volume can be about 5% squalene, about 0.5% polysorbate 80 and about 0.5% sorbitan trioleate. In weight terms, these ratios become 4.3% squalene, 0.5% polysorbate 80 and 0.48% sorbitan trioleate. This adjuvant is known as ‘MF59’ [77-79], as described in more detail in Chapter 10 of ref. 80 and chapter 12 of ref. 81. The MF59 emulsion advantageously includes citrate ions e.g. 10 mM sodium citrate buffer.
An emulsion of squalene, a tocopherol, and polysorbate 80. The emulsion may include phosphate buffered saline. It may also include Span 85 (e.g. at 1%) and/or lecithin. These emulsions may have from 2 to 10% squalene, from 2 to 10% tocopherol and from 0.3 to 3% polysorbate 80, and the weight ratio of squalene:tocopherol is preferably ≤1 as this provides a more stable emulsion. Squalene and polysorbate 80 may be present volume ratio of about 5:2 or at a weight ratio of about 11:5. Thus the three components (squalene, tocopherol, polysorbate 80) may be present at a weight ratio of 1068:1186:485 or around 55:61:25. One such emulsion (‘AS03’) can be made by dissolving Tween 80 in PBS to give a 2% solution, then mixing 90 ml of this solution with a mixture of (5 g of DL-α-tocopherol and 5 ml squalene), then microfluidising the mixture. The resulting emulsion may have submicron oil droplets e.g. with an average diameter of between 100 and 250 nm, preferably about 180 nm. The emulsion may also include a 3-de-O-acylated monophosphoryl lipid A (3d-MPL). Another useful emulsion of this type may comprise, per human dose, 0.5-10 mg squalene, 0.5-11 mg tocopherol, and 0.1-4 mg polysorbate 80 [82] e.g. in the ratios discussed above.
An emulsion of squalene, a tocopherol, and a Triton detergent (e.g. Triton X-100). The emulsion may also include a 3d-MPL (see below). It may contain a phosphate buffer.
An emulsion comprising a polysorbate (e.g. polysorbate 80), a Triton detergent (e.g. Triton X-100) and a tocopherol (e.g. an a-tocopherol succinate). The emulsion may include these three components at a mass ratio of about 75:11:10 (e.g. 750 μg/ml polysorbate 80, 110 μg/ml Triton X-100 and 100 μg/ml α-tocopherol succinate), and these concentrations should include any contribution of these components from antigens. The emulsion may also include squalene. The emulsion may also include a 3d-MPL (see below). The aqueous phase may contain a phosphate buffer.
An emulsion of squalane, polysorbate 80 and poloxamer 401 (“Pluronic™ L121”). The emulsion can be formulated in phosphate buffered saline, pH 7.4. This emulsion is a useful delivery vehicle for muramyl dipeptides, and has been used with threonyl-MDP in the “SAF-1” adjuvant [83] (0.05-1% Thr-MDP, 5% squalane, 2.5% Pluronic L121 and 0.2% polysorbate 80). It can also be used without the Thr-MDP, as in the “AF” adjuvant [84] (5% squalane, 1.25% Pluronic L121 and 0.2% polysorbate 80). Microfluidisation is preferred.
An emulsion comprising squalene, an aqueous solvent, a polyoxyethylene alkyl ether hydrophilic nonionic surfactant (e.g. polyoxyethylene (12) cetostearyl ether) and a hydrophobic nonionic surfactant (e.g. a sorbitan ester or mannide ester, such as sorbitan monoleate or ‘Span 80’). The emulsion is preferably thermoreversible and/or has at least 90% of the oil droplets (by volume) with a size less than 200 nm [85]. The emulsion may also include one or more of: alditol; a cryoprotective agent (e.g. a sugar, such as dodecylmaltoside and/or sucrose); and/or an alkylpolyglycoside. The emulsion may include a TLR4 agonist [86]. Such emulsions may be lyophilized.
An emulsion of squalene, poloxamer 105 and Abil-Care [87]. The final concentration (weight) of these components in adjuvanted vaccines are 5% squalene, 4% poloxamer 105 (pluronic polyol) and 2% Abil-Care 85 (Bis-PEG/PPG-16/16 PEG/PPG-16/16 dimethicone; caprylic/capric triglyceride).
An emulsion having from 0.5-50% of an oil, 0.1-10% of a phospholipid, and 0.05-5% of a non-ionic surfactant. As described in reference 88, preferred phospholipid components are phosphatidylcholine, phosphatidylethanolamine, phosphatidylserine, phosphatidylinositol, phosphatidylglycerol, phosphatidic acid, sphingomyelin and cardiolipin. Submicron droplet sizes are advantageous.
A submicron oil-in-water emulsion of a non-metabolisable oil (such as light mineral oil) and at least one surfactant (such as lecithin, Tween 80 or Span 80). Additives may be included, such as QuilA saponin, cholesterol, a saponin-lipophile conjugate (such as GPI-0100, described in reference 89, produced by addition of aliphatic amine to desacylsaponin via the carboxyl group of glucuronic acid), dimethyidioctadecylammonium bromide and/or N,N-dioctadecyl-N,N-bis (2-hydroxyethyl)propanediamine.
An emulsion in which a saponin (e.g. QuilA or QS21) and a sterol (e.g. a cholesterol) are associated as helical micelles [90].
An emulsion comprising a mineral oil, a non-ionic lipophilic ethoxylated fatty alcohol, and a non-ionic hydrophilic surfactant (e.g. an ethoxylated fatty alcohol and/or polyoxyethylene-polyoxypropylene block copolymer) [91].
An emulsion comprising a mineral oil, a non-ionic hydrophilic ethoxylated fatty alcohol, and a non-ionic lipophilic surfactant (e.g. an ethoxylated fatty alcohol and/or polyoxyethylene-polyoxypropylene block copolymer) [91].

In some embodiments an emulsion may be mixed with antigen extemporaneously, at the time of delivery, and thus the adjuvant and antigen may be kept separately in a packaged or distributed vaccine, ready for final formulation at the time of use. In other embodiments an emulsion is mixed with antigen during manufacture, and thus the composition is packaged in a liquid adjuvanted form, as in the FLUAD™ product. The antigen will generally be in an aqueous form, such that the vaccine is finally prepared by mixing two liquids. The volume ratio of the two liquids for mixing can vary (e.g. between 5:1 and 1:5) but is generally about 1:1. Where concentrations of components are given in the above descriptions of specific emulsions, these concentrations are typically for an undiluted composition, and the concentration after mixing with an antigen solution will thus decrease.

After the antigen and adjuvant have been mixed, haemagglutinin antigen will generally remain in aqueous solution but may distribute itself around the oil/water interface. In general, little if any haemagglutinin will enter the oil phase of the emulsion.

Where a composition includes a tocopherol, any of the α, β, γ, δ, ϵ or ξ tocopherols can be used, but α-tocopherols are preferred. The tocopherol can take several forms e.g. different salts and/or isomers. Salts include organic salts, such as succinate, acetate, nicotinate, etc. D-α-tocopherol and DL-α-tocopherol can both be used. Tocopherols are advantageously included in vaccines for use in elderly patients (e.g. aged 60 years or older) because vitamin E has been reported to have a positive effect on the immune response in this patient group [92]. They also have antioxidant properties that may help to stabilize the emulsions [93]. A preferred α-tocopherol is DL-α-tocopherol, and the preferred salt of this tocopherol is the succinate. The succinate salt has been found to cooperate with TNF-related ligands in vivo. Moreover, α-tocopherol succinate is known to be compatible with influenza vaccines and to be a useful preservative as an alternative to mercurial compounds [28].

### The Child

The invention is used to immunize children against influenza virus infection and/or disease.

The child may be aged between 0 months and 72 months, and ideally between 0 months and 36 months. Thus the child may be immunized before their 3rd or 6th birthday.

Typically the child will be at least 6 months old e.g. in the range 6-72 months old (inclusive) or in the range 6-36 months old (inclusive), or in the range 36-72 months old (inclusive). Children in these age ranges may in some embodiments be less than 30 months old, or less than 24 months old. For example, a composition may be administered to them at the age of 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 months; or at 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70 or 71 months; or at 36 or 72 months.

Where a child has been pre-immunized with an influenza B virus antigen, they are distinct from patients in general, as they are members of a subset of the general population whose immune systems have already mounted an immune response to the adjuvanted pre-immunization antigen, such that re-immunization according to the invention elicits a different immune response in the subset than in patients who have not previously mounted an immune response to the adjuvanted pre-immunization antigen. Their immune response is also different from that seen in patients who have previously mounted an immune response to the pre-immunization antigen in unadjuvanted form. The pre-immunized children will mount a booster response to the administered influenza B virus antigen, rather than a primary immune response.

### Pharmaceutical Compositions

Compositions of the invention are pharmaceutically acceptable. They may include components in addition to the antigen and adjuvant e.g. they will typically include one or more pharmaceutical carrier(s) and/or excipient(s). A thorough discussion of such components is available in ref. 94.

The composition may include preservatives such as thiomersal or 2-phenoxyethanol. It is preferred, however, that the vaccine should be substantially free from (i.e. less than 5 μg/ml) mercurial material e.g. thiomersal-free [28,95]. Vaccines containing no mercury are more preferred, and α-tocopherol succinate can be included as an alternative to mercurial compounds [28]. Preservative-free vaccines are most preferred.

To control tonicity, it is preferred to include a physiological salt, such as a sodium salt. Sodium chloride (NaCl) is preferred, which may be present at between 1 and 20 mg/ml. Other salts that may be present include potassium chloride, potassium dihydrogen phosphate, disodium phosphate dehydrate, magnesium chloride, calcium chloride, etc.

Compositions will generally have an osmolality of between 200 mOsm/kg and 400 mOsm/kg, preferably between 240-360 mOsm/kg, and will more preferably fall within the range of 290-310 mOsm/kg. Osmolality has previously been reported not to have an impact on pain caused by vaccination [96], but keeping osmolality in this range is nevertheless preferred.

Compositions may include one or more buffers. Typical buffers include: a phosphate buffer; a Tris buffer; a borate buffer; a succinate buffer; a histidine buffer (particularly with an aluminum hydroxide adjuvant); or a citrate buffer. Buffers will typically be included in the 5-20 mM range.

The pH of a composition will generally be between 5.0 and 8.1, and more typically between 6.0 and 8.0 e.g. 6.5 and 7.5, or between 7.0 and 7.8. A manufacturing process of the invention may therefore include a step of adjusting the pH of the bulk vaccine prior to packaging.

The composition is preferably sterile. The composition is preferably non-pyrogenic e.g. containing <1 EU (endotoxin unit, a standard measure) per dose, and preferably <0.1 EU per dose. The composition is preferably gluten free.

Compositions of the invention may include detergent e.g. a polyoxyethylene sorbitan ester surfactant (known as ‘Tweens’), an octoxynol (such as octoxynol-9 (Triton X-100) or t-octylphenoxypolyethoxyethanol), a cetyl trimethyl ammonium bromide (‘CTAB’), or sodium deoxycholate, particularly for a split or surface antigen vaccine. The detergent may be present only at trace amounts. Thus the vaccine may included less than 1 mg/ml of each of octoxynol-10 and polysorbate 80. Other residual components in trace amounts could be antibiotics (e.g. neomycin, kanamycin, polymyxin B).

The composition may include material for a single immunization, or may include material for multiple immunizations (i.e. a ‘multidose’ kit). The inclusion of a preservative is preferred in multidose arrangements. As an alternative (or in addition) to including a preservative in multidose compositions, the compositions may be contained in a container having an aseptic adaptor for removal of material.

Influenza vaccines are typically administered in a dosage volume (unit dose) of about 0.5 ml, although a half dose (i.e. about 0.25 ml) may be administered to children according to the invention.

Compositions and kits are preferably stored at between 2° C. and 8° C. They should not be frozen. They should ideally be kept out of direct light.

The antigen and emulsion in a composition will typically be in admixture, although they may initially be presented in the form of a kit of separate components for extemporaneous admixing. Compositions will generally be in aqueous form when administered to a subject.

### Kits of the Invention

Compositions of the invention may be prepared extemporaneously, at the time of delivery. Thus the invention provides kits including the various components ready for mixing. The kit allows the adjuvant and the antigen to be kept separately until the time of use.

The components are physically separate from each other within the kit, and this separation can be achieved in various ways. For instance, the two components may be in two separate containers, such as vials. The contents of the two vials can then be mixed e.g. by removing the contents of one vial and adding them to the other vial, or by separately removing the contents of both vials and mixing them in a third container.

In a preferred arrangement, one of the kit components is in a syringe and the other is in a container such as a vial. The syringe can be used (e.g. with a needle) to insert its contents into the second container for mixing, and the mixture can then be withdrawn into the syringe. The mixed contents of the syringe can then be administered to a patient, typically through a new sterile needle. Packing one component in a syringe eliminates the need for using a separate syringe for patient administration.

In another preferred arrangement, the two kit components are held together but separately in the same syringe e.g. a dual-chamber syringe, such as those disclosed in references 97-104 etc. When the syringe is actuated (e.g. during administration to a patient) then the contents of the two chambers are mixed. This arrangement avoids the need for a separate mixing step at the time of use.

The kit components will generally be in aqueous form. In some arrangements, a component (typically an antigen component rather than an adjuvant component) is in dry form (e.g. in a lyophilized form), with the other component being in aqueous form. The two components can be mixed in order to reactivate the dry component and give an aqueous composition for administration to a patient. A lyophilized component will typically be located within a vial rather than a syringe. Dried components may include stabilizers such as lactose, sucrose or mannitol, as well as mixtures thereof e.g. lactose/sucrose mixtures, sucrose/mannitol mixtures, etc. One possible arrangement uses an aqueous adjuvant component in a pre-filled syringe and a lyophilized antigen component in a vial.

### Packaging of Compositions or Kit Components

Suitable containers for compositions of the invention (or kit components) include vials, syringes (e.g. disposable syringes), nasal sprays, etc. These containers should be sterile.

Where a composition/component is located in a vial, the vial is preferably made of a glass or plastic material. The vial is preferably sterilized before the composition is added to it. To avoid problems with latex-sensitive patients, vials are preferably sealed with a latex-free stopper, and the absence of latex in all packaging material is preferred. The vial may include a single dose of vaccine, or it may include more than one dose (a ‘multidose’ vial) e.g. 10 doses. Preferred vials are made of colorless glass.

A vial can have a cap (e.g. a Luer lock) adapted such that a pre-filled syringe can be inserted into the cap, the contents of the syringe can be expelled into the vial (e.g. to reconstitute lyophilised material therein), and the contents of the vial can be removed back into the syringe. After removal of the syringe from the vial, a needle can then be attached and the composition can be administered to a patient. The cap is preferably located inside a seal or cover, such that the seal or cover has to be removed before the cap can be accessed. A vial may have a cap that permits aseptic removal of its contents, particularly for multidose vials.

Where a component is packaged into a syringe, the syringe may have a needle attached to it. If a needle is not attached, a separate needle may be supplied with the syringe for assembly and use. Such a needle may be sheathed. Safety needles are preferred. 1-inch 23-gauge, 1-inch 25-gauge and ⅝-inch 25-gauge needles are typical. Syringes may be provided with peel-off labels on which the lot number, influenza season and expiration date of the contents may be printed, to facilitate record keeping. The plunger in the syringe preferably has a stopper to prevent the plunger from being accidentally removed during aspiration. The syringes may have a latex rubber cap and/or plunger. Disposable syringes contain a single dose of vaccine. The syringe will generally have a tip cap to seal the tip prior to attachment of a needle, and the tip cap is preferably made of a butyl rubber. If the syringe and needle are packaged separately then the needle is preferably fitted with a butyl rubber shield. Useful syringes are those marketed under the trade name “Tip-Lok”™.

Containers may be marked to show a half-dose volume e.g. to facilitate delivery to children. For instance, a syringe containing a 0.5 ml dose may have a mark showing a 0.25 ml volume.

Where a glass container (e.g. a syringe or a vial) is used, then it is preferred to use a container made from a borosilicate glass rather than from a soda lime glass.

A kit or composition may be packaged (e.g. in the same box) with a leaflet including details of the vaccine e.g. instructions for administration, details of the antigens within the vaccine, etc. The instructions may also contain warnings e.g. to keep a solution of adrenaline readily available in case of anaphylactic reaction following vaccination, etc.

### Methods of Treatment, and Administration of the Vaccine

Compositions of the invention are suitable for administration to human patients, and the invention provides a method of raising an immune response in a patient, comprising the step of administering a composition of the invention to the patient. As described above, the patient is a child.

The invention also provides a kit or composition of the invention for use as a medicament. The invention also provides the medical uses discussed above.

These methods and uses will generally be used to generate an antibody response, preferably a protective antibody response. Methods for assessing antibody responses, neutralizing capability and protection after influenza virus vaccination are well known in the art. Human studies have shown that antibody titers against hemagglutinin of human influenza virus are correlated with protection (a serum sample hemagglutination-inhibition titer of about 30-40 gives around 50% protection from infection by a homologous virus) [105]. Antibody responses are typically measured by hemagglutination inhibition (HI), by microneutralization (Micro-NT), by single radial immunodiffusion (SRID), and/or by single radial hemolysis (SRH). These assay techniques are well known in the art.

Compositions of the invention can be administered in various ways. The most preferred immunization route is by intramuscular injection (e.g. into the arm or leg), but other available routes include subcutaneous injection, intranasal [106-108], oral [109], intradermal [110,111], transcutaneous, transdermal [112], etc.

Preferred compositions of the invention will satisfy 1, 2 or 3 of the CPMP criteria for adult efficacy for each influenza strain, even though they are administered to children. These criteria are: (1) ≥70% seroprotection; (2) ≥40% seroconversion or significant increase; and/or (3) a GMT increase of ≥2.5-fold. In elderly (>60 years), these criteria are: (1) ≥60% seroprotection; (2) ≥30% seroconversion; and/or (3) a GMT increase of ≥2-fold. These criteria are based on open label studies with at least 50 patients.

The invention is particularly useful for raising immune responses that are protective against different influenza B virus strains. The invention may also be effective against drifted (mismatched) influenza A virus strains (particularly drifted A/H3N2 strains).

Treatment with compositions of the invention can be by a single dose schedule or a multiple dose schedule. Thus, in any particular influenza season (e.g. in a given 12 month period, typically in autumn or winter) a patient may receive a single dose of a composition of the invention or more than one dose of composition of the invention (e.g. two doses). Where treatment comprises administration of two or more doses of compositions of the invention, each dose will generally not be given at substantially the same time i.e. they will not be administered during the same visit to a vaccination centre. The time between successive administration of compositions of the invention is typically at least n days, where n is selected from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 42, 49, 56 or more. Typically, two doses are administered at least 1 week apart (e.g. about 2 weeks, about 3 weeks, about 4 weeks, about 6 weeks, about 8 weeks, about 12 weeks, about 16 weeks, etc.). Giving two doses separated by from 25-30 days (e.g. 28 days) is particularly useful. The time between doses will typically be no longer than 6 months. The doses may be given about 4 weeks apart from each other e.g. at day 0 and then at about day 28. Separation of dosing in this way has been found to give good immune responses.

Where compositions of the invention are used in a primary immunization schedule, dose(s) with compositions of the invention are followed by administration of one or more booster vaccines (e.g. 1, 2, 3, or more booster vaccines). The booster vaccine comprises one or more influenza virus B antigens from a different strain or lineage to the influenza B antigen in the composition(s) of the invention. The booster vaccine can be adjuvanted or unadjuvanted. Suitable timing between priming and administration of booster vaccine can be routinely determined. The time between administration of a priming dose and administration of a booster vaccine is typically at least p months, where p is selected from 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, or more. Ideally, p is 9 or more, and may be within the range of 9-30.

Where compositions of the invention are used in a booster immunization schedule, the patient has been has been pre-immunized with an influenza B virus antigen from a different strain or lineage of influenza B virus e.g. as part of a previous seasonal vaccination regimen.

In a multiple dose schedule the various doses may be given by the same or different routes e.g. a parenteral prime and mucosal boost, a mucosal prime and parenteral boost, etc. Typically they will be given by the same route. Vaccines produced by the invention may be administered to patients at substantially the same time as (e.g. during the same medical consultation or visit to a healthcare professional or vaccination centre) other vaccines e.g. at substantially the same time as a measles vaccine, a mumps vaccine, a rubella vaccine, a MMR vaccine, a varicella vaccine, a MMRV vaccine, a diphtheria vaccine, a tetanus vaccine, a pertussis vaccine, a DTP vaccine, a conjugated H. influenzae type b vaccine, an inactivated poliovirus vaccine, a hepatitis B virus vaccine, a meningococcal conjugate vaccine (such as a tetravalent A-C-W135-Y vaccine), a pneumococcal conjugate vaccine, etc.

Similarly, vaccines of the invention may be administered to patients at substantially the same time as (e.g. during the same medical consultation or visit to a healthcare professional) an antiviral compound, and in particular an antiviral compound active against influenza virus (e.g. oseltamivir and/or zanamivir). These antivirals include neuraminidase inhibitors, such as a (3R,4R,5S)-4-acetylamino-5-amino-3(1-ethylpropoxy)-1-cyclohexene-1-carboxylic acid or 5-(acetylamino)-4-[(aminoiminomethyl)-amino]-2,6-anhydro-3,4,5-trideoxy-D-glycero-D-galactonon-2-enonic acid, including esters thereof (e.g. the ethyl esters) and salts thereof (e.g. the phosphate salts). A preferred antiviral is (3R,4R,5S)-4-acetylamino-5-amino-3(1-ethylpropoxy)-1-cyclohexene-1-carboxylic acid, ethyl ester, phosphate (1:1), also known as oseltamivir phosphate (TAMIFLU™).

### General

The term “comprising” encompasses “including” as well as “consisting” e.g. a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in relation to a numerical value x means, for example, x±10%.

Unless specifically stated, a process comprising a step of mixing two or more components does not require any specific order of mixing. Thus components can be mixed in any order. Where there are three components then two components can be combined with each other, and then the combination may be combined with the third component, etc.

Where animal (and particularly bovine) materials are used in the culture of cells, they should be obtained from sources that are free from transmissible spongiform encaphalopathies (TSEs), and in particular free from bovine spongiform encephalopathy (BSE). Overall, it is preferred to culture cells in the total absence of animal-derived materials.

Where a compound is administered to the body as part of a composition then that compound may alternatively be replaced by a suitable prodrug.

Where a cell substrate is used for reassortment or reverse genetics procedures, it is preferably one that has been approved for use in human vaccine production e.g. as in Ph Eur general chapter 5.2.3.

### MODES FOR CARRYING OUT THE INVENTION

Healthy children (6 to <36 months of age) never being previously vaccinated against influenza were invited to participate in the trial. Subjects were randomized to receive one of the two trivalent inactivated influenza vaccines: a subunit vaccine adjuvanted with MF59.™. (FLUAD), or a non-adjuvanted split vaccine (Influsplit SSW). Two doses, 0.25 ml each, were given intramuscularly in the deltoid region of the non-dominant arm or, if the deltoid mass was insufficient, in the anterolateral aspect of the thigh. The second vaccination was four weeks after the first.

The antigenic composition of the vaccine was in agreement with WHO recommendations for the Northern Hemisphere during the 2008/09 influenza season. Each dose of 0.25 ml vaccine contained 7.5 μg of each of the three influenza antigens: A/Brisbane/59/2007 (H1N1)-like virus, A/Brisbane/10/2007 (H.sub.3N.sub.2)-like virus, B/Florida/4/2006-like virus. B/Florida/4/2006-like virus is a Victoria lineage influenza B virus.

Children who had been primed were offered to receive a booster dose of the adjuvanted vaccine or unadjuvanted split vaccine approximately two years later. Healthy children who had been primed with two intramusluclar (IM) doses for the 2008/09 season were re-randomised and received a third intramuscular dose of the respective adjuvanted (Fluad) or non-adjuvanted (Agrippal) vaccine (2010/11 Northern Hemisphere vaccine formulation) approximately two years after the first dose. The antigenic composition of the booster vaccines was in agreement with WHO recommendations for the Northern Hemisphere during the 2010/11 influenza season. Each dose of 0.25 ml vaccines contained 7.5 μg of each of the three influenza antigens: A/California/7/2009 (H1N1)-like virus, A/Perth/16/2009 (H.sub.3N.sub.2)-like virus, B/Brisbane/60/2008-like virus. B/Brisbane/60/2008-like virus is a Yamagata lineage influenza B virus. Therefore, for the 2010/11 season, all three influenza strains changed compared to the vaccine formulation of the booster campaign. The influenza B virus antigen was from a different lineage.

For the influenza B virus antigen, baseline antibody titers (i.e. GMT Day 1) were higher in children receiving adjuvanted (FLUAD) vaccine, confirming a better persistence of immunogenicity after priming than with a non-adjuvanted vaccine.

Three weeks after receiving the booster vaccination, children primed with (non-adjuvanted) vaccine gave a mediocre immune response to the single administration of influenza B virus antigen in the booster, which was from a different lineage. The mediocre response was similar to unprimed controls, irrespective of whether the booster vaccine comprised an adjuvant or not, but children who received adjuvanted booster performed slightly better. This result is supports the current ACIP recommendation for a two-dose vaccination regimen in immunologically naïve children e.g. when there has been a change in influenza B virus lineage.

Surprisingly, children primed with oil-in-water emulsion-adjuvanted vaccine gave a strong immune response to the influenza B virus antigen in the booster, even though it was from a different lineage (and strain) of influenza B virus. This strong cross-lineage immune response was achieved after only a single booster vaccination and was independent of whether the booster vaccine comprised an adjuvant.

Thus, immunogenic priming with a composition comprising influenza B virus antigen and an oil-in-water emulsion (e.g. a seasonal influenza vaccine such as FLUAD) primes an immune response to influenza B virus antigen from a different lineage. Therefore, a child that has been primed with an immunogenic composition according to the invention (such as FLUAD) may require only one booster vaccination if there has been a change in the lineage of influenza B virus. The invention therefore avoids the second vaccination currently recommended by the ACIP.

These data indicate the importance and advantage of priming with an oil-in-water emulsion-adjuvanted vaccine comprising influenza B in children, especially those less than 72 months old. Immunogenic priming of children with influenza B vaccine adjuvanted with an oil-in-water emulsion primes an immune response to a booster vaccine comprising influenza B virus antigen from a different strain or lineage, irrespective of whether the booster comprises an adjuvant.

TABLE 1
Geometric mean titers (GMTs) obtained from this study. Children were primed with Fluad (adjuvanted)
or Influsplit SSW (non-adjuvanted) comprising 2008/09 Northern hemisphere winter season influenza
antigens. Priming controls received MenC vaccine (Encepur). Approximately two years later, children
received a booster vaccination with adjuvanted influenza vaccine (Fluad) or non-adjuvanted influenza
vaccine (Agrippal) comprising 2010/11 Northern hemisphere winter season influenza antigens. Priming
controls received a booster of only adjuvanted influenza (Fluad).

| | | A/California/2009 | | A/Perth/2009 | | B/Brisbane/ | |
|---|---|---|---|---|---|---|---|
| | | (A/H1N1) | | (A/H3N2) | | 2008 | |
| | | Fluad | Agrippal | Fluad | Agrippal | Fluad | Agrippal |
| | | N = 48 | N = 30 | N = 48 | N = 30 | N = 48 | N = 30 |
| FLUAD | GMT | 61 | 45 | 129 | 103 | 8.53 | 11 |
| priming | Day 1 | (42-89) | (28-72) | (84-197) | (60-177) | (6.42-11) | (7.48-15) |
| | GMT | 1157 | 502 | 1836 | 770 | 127 | 111 |
| | Day 22 | (797-1679) | (313-804) | (1460-2310) | (576-1029) | (94-171) | (76-162) |
| | | N = 57 | N = 31 | N = 57 | N = 31 | N = 57 | N = 31 |
| (Influsplit SSW) | GMT | 54 | 53 | 45 | 26 | 6.69 | 6.11 |
| priming | Day 1 | (38-77) | (33-85) | (27-74) | (13-51) | (5.56-8.05) | (4.76-7.86) |
| | GMT | 1394 | 732 | 745 | 265 | 45 | 26 |
| | Day 22 | (1071-1814) | (512-1046) | (544-1021) | (173-406) | (32-64) | (16-41) |
| | | N = 27 | | N = 27 | | N = 27 | |
| Unprimed | GMT | 63 | | 122 | | 6.63 | |
| Control | Day 1 | (38-103) | | (61-244) | | (4.88-9.01) | |
| | GMT | 1720 | | 1592 | | 45 | |
| | Day 22 | (1174-2518) | | (1023-2477) | | (24-86) | |

It will be understood that the invention has been described by way of example only and modifications may be made whilst remaining within the scope and spirit of the invention.

### REFERENCES

[1] Vaccines. (eds. Plotkin & Orenstein). 4th edition, 2004, ISBN: 0-7216-9688-0.

[2] Neuzil et al. (2000) N Engl J Med 342:225-31.

[3] Peltola et al. (2003) Clin Infect Dis 36:299-305.

[4] Heikkinen et al. (2004) J Infect Dis 190:1369-73.

[5] Izurieta et al. (2000) N Engl J Med 342:232-39.

[6] Poehling et al. (2006) N Engl J Med 355:31-40.

[7] Iskander et al. (2007) Current Opin Infect Dis 20:259-263.

[8] Ghendon et al. (2006) Epidemiol Infect 134:71-78.

[9] Principi et al. (2003) Pediatr Infect Dis J 22(10 Suppl):S207-10.

[10] Fiore et al. (2008) MMWR Early Release 2008;57:1-60.

[11] ECDC. Technical report on the scientific panel on vaccines and immunization. Infant and children seasonal immunization against influenza on a routine basis during inter-pandemic period. Stockholm, January 2007.

[12] Demicheli et al. (2006) The Cochrane collaboration. Vaccines for preventing influenza in healthy children (Review). The Cochrane library, 2006, issue 3. Available at: www.thecochranelibrary.com

[13] Walter et al. (2006) Pediatrics 118:e570-78.

[14] Mitchell et al. (2005) Pediatr Infect Dis J 10:925-26.

[15] Centers for Disease Control and Prevention. Prevention and control of influenza. MMWR Early Release 2007;56.June 29:1-53.

[16] Treanor (2004). N Engl J Med. 350(3): 218-20.

[17] Ferguson, et al. (2003). Nature. 422(6930): 428-33.

[18] Koelle et al. (2006). Science 314(5807): 1898-903.

[19] Skowronski et al. (2007). Vaccine 25(15): 2842-51.

[20] Louie et al. (2006). Pediatrics. 117(4): e610-8.

[21] WO96/37624.

[22] WO98/46262.

[23] WO2007/085969.

[24] WO02/28422.

[25] WO02/067983.

[26] WO02/074336.

[27] WO01/21151.

[28] WO02/097072.

[29] WO2005/113756.

[30] Huckriede et al. (2003) Methods Enzymol 373:74-91.

[31] Treanor et al. (1996) J Infect Dis 173:1467-70.

[32] Keitel et al. (1996) Clin Diagn Lab Immunol 3:507-10.

[33] Rota et al. (1992) J Gen Virol 73:2737-42.

[34] GenBank sequence GI:325176.

[35] Hoffmann et al. (2002) Vaccine 20:3165-3170.

[36 ] Subbarao et al. (2003) Virology 305:192-200.

[37] Liu et al. (2003) Virology 314:580-590.

[38] Ozaki et al. (2004) J. Virol. 78:1851-1857.

[39] Webby et al. (2004) Lancet 363:1099-1103.

[40] WO00/60050.

[41] WO01/04333.

[42] U.S. Pat. No. 6,649,372.

[43] Neumann et al. (2005) Proc Nall Acad Sci USA 102:16825-9.

[44] WO2006/067211.

[45] WO01/83794.

[46] Hoffmann et al. (2000) Virology 267(2):310-7.

[47] Herlocher et al. (2004) J Infect Dis 190(9):1627-30.

[48] Le et al. (2005) Nature 437(7062):1108.

[49] Kistner et al. (1998) Vaccine 16:960-8.

[50] Kistner et al. (1999) Dev Biol Stand 98:101-110.

[51] Bruhl et al. (2000) Vaccine 19:1149-58.

[52] Pau et al. (2001) Vaccine 19:2716-21.

[53] http://www.atcc.org/

[54] ttp://locus.umdnj.edu/

[55] WO03/076601.

[56] WO2005/042728.

[57] WO03/043415.

[58] WO01/85938.

[59] WO2006/108846.

[60] WO97/37000.

[61] Brands et al. (1999) Dev Biol Stand 98:93-100.

[62] Halperin et al. (2002) Vaccine 20:1240-7.

[63] Tree et al. (2001) Vaccine 19:3444-50.

[64] EP-A-1260581 (WO01/64846).

[65] WO2006/071563.

[66] WO2005/113758.

[67] WO97/37001.

[68] WO2006/027698.

[69] EP-B-0870508.

[70] U.S. Pat. No. 5,948,410.

[71] Lundblad (2001) Biotechnology and Applied Biochemistry 34:195-197.

[72] Guidance for Industry: Bioanalytical Method Validation. U.S. Department of Health and Human Services Food and Drug Administration Center for Drug Evaluation and Research (CDER) Center for Veterinary Medicine (CVM). May 2001.

[73] Ji et al. (2002) Biotechniques. 32:1162-7.

[74] Briggs (1991) J Parenter Sci Technol. 45:7-12.

[75] Lahijani et al. (1998) Hum Gene Ther. 9:1173-80.

[76] Lokteff et al. (2001) Biologicals. 29:123-32.

[77] WO90/14837.

[78] Podda & Del Giudice (2003) Expert Rev Vaccines 2:197-203.

[79] Podda (2001) Vaccine 19: 2673-2680.

[80] Vaccine Design: The Subunit and Adjuvant Approach (eds. Powell & Newman) Plenum Press 1995 (ISBN 0-306-44867-X).

[81] Vaccine Adjuvants: Preparation Methods and Research Protocols (Volume 42 of Methods in Molecular Medicine series). ISBN: 1-59259-083-7. Ed. O'Hagan.

[82] WO2008/043774.

[83] Allison & Byars (1992) Res Immunol 143:519-25.

[84] Hariharan et al. (1995) Cancer Res 55:3486-9.

[85] US-2007/014805.

[86] US-2007/0191314.

[87] Suli et al. (2004) Vaccine 22(25-26):3464-9.

[88] WO95/11700.

[89] U.S. Pat. No. 6,080,725.

[90] WO2005/097181.

[91] WO2006/113373.

[92] Han et al. (2005) Impact of Vitamin E on Immune Function and Infectious Diseases in the Aged at Nutrition, Immune functions and Health EuroConference, Paris, 9-10 Jun. 2005.

[93] U.S. Pat. No. 6,630,161.

[94] Gennaro (2000) Remington: The Science and Practice of Pharmacy. 20th edition, ISBN: 0683306472.

[95] Banzhoff (2000) Immunology Letters 71:91-96.

[96] Nony et al. (2001) Vaccine 27:3645-51.

[97] WO2005/089837.

[98] U.S. Pat. No. 6,692,468.

[99] WO00/07647.

[100] WO99/17820.

[101] U.S. Pat. No. 5,971,953.

[102] U.S. Pat. No. 4,060,082.

[103] EP-A-0520618.

[104] WO98/01174.

[105] Potter & Oxford (1979) Br Med Bull 35: 69-75.

[106] Greenbaum et al. (2004) Vaccine 22:2566-77.

[107] Zurbriggen et al. (2003) Expert Rev Vaccines 2:295-304.

[108] Piascik (2003) J Am Pharm Assoc (Wash DC). 43:728-30.

[109] Mann et al. (2004) Vaccine 22:2425-9.

[110] Halperin et al. (1979) Am J Public Health 69:1247-50.

[111] Herbert et al. (1979) J Infect Dis 140:234-8.

[112] Chen et al. (2003) Vaccine 21:2830-6.

1-16. (canceled)

17: A method for immunizing a subject, the method comprising a step of:
administering to a subject a single booster dose of an immunogenic composition comprising an antigen from a second influenza B virus strain,
wherein the subject is up to 8 years of age and has received one priming dose of an immunogenic composition comprising an antigen from a first influenza B virus strain and an adjuvant comprising an oil-in-water emulsion,
wherein the first influenza B virus strain and the second influenza B virus strain are heterologous strains or in different lineages,
and wherein the single booster dose is administered at least 9 months after the priming dose.

18: The method of claim 17, wherein the single booster dose is administered within 9-30 months after the priming dose.

19: The method of claim 17, wherein the single booster dose does not comprise an adjuvant.

20: The method claim 17, wherein the majority of oil droplets in the oil-in-water emulsion have a diameter of less than 1 μm.

21: The method claim 17, wherein the oil-in-water emulsion comprises squalene.

22: The method of claim 17, wherein the first influenza B virus strain and the second influenza B virus strain are in different lineages.

23: The method of claim 17, wherein the subject is between 15 and 72 months old.

24: The method of claim 17, wherein the priming dose and/or the single booster dose includes antigen from an H1N1 influenza A virus and antigen from an H3N2-type influenza A virus.

25: The method of claim 17, wherein the immunogenic composition of the single booster dose is a whole virus vaccine, a split virus vaccine, or a subunit vaccine.

26: The method of claim 17, further comprising a step of: administering to the subject, prior to the administration of the single booster dose, the at least one priming dose of an immunogenic composition comprising an antigen from a first influenza B virus strain and an adjuvant comprising an oil-in-water emulsion.

27: The method of claim 26, wherein the priming dose and/or the single booster dose includes antigen from an H1N1 influenza A virus and antigen from an H3N2-type influenza A virus.

28: The method of claim 26, wherein the majority of oil droplets in the oil-in-water emulsion have a diameter of less than 1 μm.

29: The method of claim 26, wherein the oil-in-water emulsion comprises squalene.

30: The method of claim 26, wherein the immunogenic composition of the single booster dose is a whole virus vaccine, a split virus vaccine, or a subunit vaccine.

31: The method of claim 17, wherein the single booster dose is administered at least 23 months after the priming dose.

32: The method of claim 31, wherein the single booster dose is administered about two years after the priming dose.

33: A method for immunizing a subject, the method comprising a step of:
administering to a subject a single booster dose of an immunogenic composition comprising an antigen from a second influenza B virus strain,
wherein the subject is up to 8 years of age and has received one priming dose of an immunogenic composition comprising an antigen from a first influenza B virus strain and an adjuvant comprising an oil-in-water emulsion,
wherein the first influenza B virus strain and the second influenza B virus strain are heterologous strains or in different lineages,
wherein the single booster dose is administered at least 9 months after the priming dose,
and wherein the immunogenic composition is effective in achieving a gross mean titer of ≥2.5 fold in a population of at least 50 subjects of 8 years of age when administered as claimed.

34: The method of claim 33, wherein the subject is between 15 and 72 months old.

35: The method of claim 33, wherein the immunogenic composition of the single booster dose is a whole virus vaccine, a split virus vaccine, or a subunit vaccine.

36: The method of claim 33, wherein the single booster dose is administered at least 23 months after the priming dose.
